(19) United States
(12) Patent Application Publication
(10) Pub. No.: US 2016/0058692 A1
(43) Pub. Date: Mar. 3, 2016

(54) USE OF ACTIVATORS OF AUTOPHAGY OF SKIN CELLS FOR ENCOURAGING CELLULAR AND TISSUE LONGEVITY OF THE SKIN

(71) Applicant: SOCIETE INDUSTRIELLE LIMOUSINE D'APPLICATION BIOLOGIQUE, Objat (FR)

(72) Inventor: Jean PAUFIQUE, OBJAT (FR)

(21) Appl. No.: 14/784,249

(22) PCT Filed: Apr. 11, 2014
(86) PCT No.: PCT/FR2014/050888

§ 371 Date: Oct. 13, 2015

(30) Foreign Application Priority Data
Apr. 11, 2013 (FR) 13 532 98

(51) Int. Cl. A61K 8/97; A61Q 19/08

(52) U.S. Cl. CPC A61K 8/97; A61Q 19/08; A61K 2800/782

(57) ABSTRACT

The use, in a cosmetic composition, of an effective quantity of at least one activator of autophagy of skin cells as a cosmetic active substance, the active substance and/or the composition being used to increase the cellular and tissue longevity of the skin. Also a cosmetic method for treating the skin in order to improve the cellular and tissue longevity of the skin.

This invention relates to the use of an activator of skin-cell autophagy for improving cellular and tissue longevity of the skin. The object of the invention is also specifically a cosmetic skin treatment process for promoting cellular and tissue longevity of the skin, comprising the application on the skin of an activator of skin-cell autophagy.

Aging is a complex process that depends on genetic determinism, epigenetic mechanisms, and various external influences, such as the sun, atmospheric pollution, food, lifestyle, etc. The longevity and the decline of cells and tissues of the skin are therefore under the influence of numerous factors.

To effectively prevent the aging of the skin, including premature aging, and to limit the appearance of negative effects that result therefrom, it is important to delay the appearance of the phenomena of cellular senescence and to limit tissue degeneration.

This is why the objective of this invention is to find a cosmetic solution that is capable of acting on molecular mechanisms that are strongly involved in cellular and tissue longevity and thus to limit and to push back the appearance of the signs of aging of the skin.

To respond to this, this invention proposes using an activator of skin-cell autophagy.

Actually, surprisingly enough, the application on the skin of an activator of skin-cell autophagy makes it possible to increase cellular and tissue longevity of the skin.

The object of the invention is therefore the use in a cosmetic composition of at least one activator of skin-cell autophagy as an active ingredient intended to increase cellular and tissue longevity of the skin.

According to the invention, the use of an activator of the autophagy makes it possible to delay the appearance of the phenomena of cellular senescence of the skin and to limit the tissue degeneration by acting on molecular mechanisms that are strongly involved in cellular and tissue longevity. It makes it possible to preserve the functionality of the tissues of the skin and the appearance of the signs of aging in the area of the skin.

According to a particular aspect, the object of the invention is specifically a cosmetic skin treatment process for increasing cellular and tissue longevity, comprising the topical application on the skin of an active ingredient that activates skin-cell autophagy.

This invention is now described in detail.

Autophagy is a mechanism for recycling and detoxifying cellular components and organelles. Autophagy makes it possible in particular to regulate, repair and eliminate the proteins of long service life in the cells, thus ensuring a control during the differentiation and aging of the human skin.

On the cellular plane, the autophagy mechanism comprises four stages: the initiation, the formation of an initial vacuole, called an autophagosome, which sequesters the cytoplasmic material, the maturation of the autophagosome in a degradative vacuole, and the fusion with the lysosome, until the sequestered material is degraded.

Activator or stimulator of autophagy in terms of the invention is defined as a molecule or an active ingredient, or a mixture of molecules or active ingredients, capable of activating the mechanism of the autophagy.

The object of this invention is therefore an activator of skin-cell autophagy for its application as an active ingredient in a composition with topical application on the skin, with said active ingredient and/or said composition being intended to increase cellular and tissue longevity of the skin.

In particular, the object of the invention is the use of an active ingredient that activates skin-cell autophagy as an active ingredient in a composition with topical application, with said active ingredient and/or said composition being intended to act on the molecular mechanisms involved in cellular and tissue longevity of the skin, in particular:
To limit the accumulation of lipofuscin in the skin cells, and
To limit degradation of the cellular matrix induced by the metalloproteinases 1 (MMP-1).

Lipofuscin, also called “age pigment,” is a non-degradable, autofluorescent compound that gradually accumulates during repeated stress or with age and that impedes proper operation of the cell. In the area of cutaneous tissue, the deposits of lipofuscin contribute to the appearance of age spots that frequently characterize skin that is aged or over-exposed to the sun. Its accumulation is associated with a reduction of cellular longevity.

According to the invention, however, the use on the skin of an activator of skin-cell autophagy makes it possible to limit the accumulation of lipofuscin in the skin cells and consequently to increase cellular and tissue longevity of the skin.

Furthermore, the synthesis of MMP-1, matrix metalloproteinase responsible for the degradation of collagen I and III fibers, is increased in the skin cells during the aging of the skin. This degradation of the three-dimensional matrix network of the skin cells is reflected on the surface of the skin by a release, a loss of firmness, and the appearance of wrinkles.

According to the invention, however, the use on the skin of an activator of skin-cell autophagy makes it possible to inhibit or to limit the synthesis of MMP-1 in the skin cells and consequently to reduce the degradation of the extracellular matrix and therefore to increase cellular and tissue longevity of the skin.

These actions are reflected visually by a reduction of the signs of aging of the skin, in particular a reduction of wrinkles and an improvement of the homogeneity of the complexion.

Preferably, the stimulator of the autophagic activity of the skin cells that is useful according to the invention is a synthetic molecule or an active ingredient that is obtained from at least one plant, one alga or one yeast.

The activator of autophagy can be, for example, an active ingredient that is capable of activating skin-cell autophagy, obtained from Prunus cerasus or Metschnikowia hawaiiensis.

According to one aspect of the invention, the activator of skin-cell autophagy is able to be selected by a test that is carried out on skin-cell cultures as described in the patent FR-2,939,316. The activator of autophagy is therefore able to be selected by a test that is carried out on skin-cell cultures comprising the following stages:
Culture of skin cells, keratinocytes, or fibroblasts, in a suitable culture medium,
Removal of the culture medium and replacement by a culture medium comprising a stressing agent inducing a nutritive and/or oxidative stress,
Addition of an active ingredient obtained from a plant, alga or yeast or a synthetic molecule to be tested within the culture medium comprising the active ingredient, and
Analysis of the expression of MAP-LC3, ATG5-12, phosphorylated mTOR, phosphorylated p53 protein, expression of the DRAM protein and/or AMPK by said cells, and comparison of the results with those obtained on skin-cell cultures that are not treated with the active ingredient that is to be tested.

The activators of autophagy for use according to the invention can be incorporated in cosmetic compositions in various galenical forms, adapted to administration by cutaneous topical means.

These compositions can come in particular in the form of creams, oil-in-water emulsions, water-in-oil emulsions, multiple emulsions, solutions, suspensions or powders. They can be more or less fluid and have the appearance of a cream, a lotion, a milk, a serum, an ointment, a gel, a paste, or a foam, or in solid form.

These compositions contain between 0.01 and 15% by weight of activators of skin-cell autophagy according to this invention, preferably between 0.5% and 4%.

According to another aspect, the object of the invention is also a cosmetic skin treatment process for increasing cellular and tissue longevity, comprising the topical application on the skin of an activator of skin-cell autophagy or a composition that contains an activator of skin-cell autophagy. In particular, the object of the invention is a cosmetic process for treatment of photoexposed skin to delay the appearance of aging, comprising the topical application on the skin of an activator of skin-cell autophagy or a composition that contains an activator of skin-cell autophagy.

To illustrate the invention, multiple activators of autophagy have been selected and tested for studying their capacity for increasing cellular and tissue longevity of the skin.

### I. Examples of Activators of Autophagy

### I.1. Activator of Autophagy Obtained from Prunus cerasus

One example of an activator of autophagy that is useful according to the invention is an active ingredient that is obtained from the fruit-bearing peduncle of Prunus cesarus that responds positively to the test carried out on skin-cell cultures as described in the patent FR-2,939,316.

Such an active ingredient can be obtained by implementing the following stages:
Solubilization of fruit-bearing peduncle powder from a cherry tree in water,
Enzymatic hydrolyses,
Separation of soluble and insoluble phases and recovery of the soluble phase,
Enzymatic deactivation by heat treatment,
Filtration and purification of oligosaccharides,
Concentration of the active oligosaccharide fraction,
Filtration and sterilizing filtration.

The active ingredient contains approximately 40% of oligosaccharides of a size of between 180 Da and 1,300 Da.

### I.2 Activator of Autophagy Obtained from Metschnikowia hawaiiensis

Another example of a useful activator of autophagy according to the invention is an active ingredient that is obtained from Metschnikowia hawaiiensis that responds positively to the test carried out on skin-cell cultures as described in the patent FR-2,939,316.

Such an active ingredient can be obtained by implementing the following stages:
Solubilization of Metschnikowia hawaiiensis powder in water,
Hydrolyses,
Filtration,
Filtration and purification for recovery of peptides,
Filtration and sterilizing filtration.

The active ingredient contains approximately 50% of peptides of a size of between 243 Da and 5,000 Da.

### II. Effectiveness of the Activators of Autophagy on the Formation of Lysosomes

The objective of this study is to evaluate the effect of active ingredients on the production of lysosomes, organelles involved in the autophagic mechanism of the cell.

This study was carried out by flow cytometry on normal human fibroblasts subjected to moderate and repeated treatments with hydrogen peroxide (H₂O₂) making it possible to induce cellular senescence.

The operating procedure is described below.

On D0, the normal human fibroblasts are inoculated in the suitable medium.

For several days, the fibroblasts are treated:
Non-Stressed Fibroblasts: the human fibroblasts are inoculated in the culture medium with or without the presence of one of the active ingredients.
Control Stressed Fibroblasts: the human fibroblasts are inoculated in the culture medium and then treated with the H₂O₂ solution. This repeated H₂O₂ treatment makes it possible to induce the cellular senescence state characterized by a growth ratio and a β-galactosidase activity.
Stressed fibroblasts treated with the active ingredients of Examples I. 1 and I. 2: the human fibroblasts are inoculated in the culture medium in the presence of active ingredients and then treated with the H₂O₂ solution.

A specific marking of the lysosomes is carried out by fluorescence.

The analysis of fluorescent marking is carried out by flow cytometry. The intensity of green fluorescence is proportional to the quantity of lysosomes. The greater the fluorescent intensity, the greater the formation of lysosomes.

The results are presented in the table below:

| | Effectiveness/Control Stressed Fibroblasts |
|---|---|
| | (%) |
| Stressed Control | 0 |
| 1% Prunus cerasus Extract | +45% |
| 1% Metschnikowia | +99% |
| hawaiiensis Extract | |

Tested at 1% on stressed human fibroblasts, the active ingredients extracted from the cherry tree and extracted from Metschnikowia hawaiiensis significantly boost the production of lysosomes by 45% and 99% in comparison to the control stressed fibroblasts.

By increasing the production of lysosomes in the cell cultures, these active ingredients activate the autophagy mechanism.

### III. Examples of Compositions Including These Activators of Autophagy

### III. 1 Night Cream

| A. | Water | Enough to produce 100% |
|---|---|---|
| | Propylene glycol | 10% |
| | Carbopol 2050 (Noveon) | 0.2% |
| B. | DUB MCT 5545 (Stéarinerie Dubois) | 10% |
| | DUB Liquid 85 (Stéarinerie Dubois) | 6% |
| | DUB Wax A (Stéarinerie Dubois) | 2% |
| | DUB G1218A (Stéarinerie Dubois) | 3% |
| | DUB 1632 (Stéarinerie Dubois) | 2% |
| | DC 200 (Dow Corning) | 2% |
| C. | Preservative | 1% |
| | Active ingredient of Example I. 2 | 3% |
| D. | NaOH | 0.5% |

This composition is an emulsified gel, with pH 5.

It is obtained by implementing the following stages:
Mixing A, heating it to 60° C., and dispersing the carbopol thoroughly while being stirred mechanically
Heating A and B to 80° C., while being stirred mechanically
Emulsifying B in A under an emulsifying agent
At 40° C., adding C in order, homogenizing
Then at 30° C., adjusting the pH with C
Continuing homogenization until the emulsion is uniform.

### III. 2. Anti-Aging Day Cream

| | | |
|---|---|---|
| A. | Water | Enough to produce 100% |
| | Carbopol Ultrez 20 (Noveon) | 0.3% |
| | Glycerol | 3% |
| | EDTA | 0.2% |
| B. | Sophim MC30 (Sophim) | 6% |
| | DUB MCT 5545 (Stéarinerie Dubois) | 5% |
| | Karite butter (Sictia) | 3% |
| | Sterol CC/595 (Cesalpina) | 3% |
| | Ritapro 165 (Rita) | 3% |
| | DC 345 (Dow Corning) | 1% |
| | DUB zenoate (Stéarinerie Dubois) | 2% |
| C. | Preservative | 1% |
| | Active ingredient of Example I. 1 | 3% |
| D. | TEA | Enough to produce pH 5.3 |

This composition is emulsified gel, with pH 5.3.

It is obtained by implementing the following stages:
Mixing A, mixing B,
Heating A and B while being stirred mechanically at 80° C.,
Emulsifying B in A while being stirred,
At 40° C., adding C in order, homogenizing,
Continuing the homogenization by adding D until the mixture is uniform and allowing it to cool while being stirred until 30° C. is reached.

### III. 3. Serum

| | | |
|---|---|---|
| A. | Water | Enough to produce 100% |
| | Glycerol | 3% |
| | Carbopol ETD 2050 (Noveon) | 0.3% |
| | Butylene glycol | 5% |
| B. | DUB ININ (Stéarinerie Dubois) | 3% |
| | Arlamol E (Noveon) | 3% |
| | DUB PTIS (Stéarinerie Dubois) | 2% |
| | DUB PTCC (Stéarinerie Dubois) | 3.3% |
| C. | Preservative | 1% |
| | Active ingredient of Example I. 2 | 3% |
| D. | NaOH- | Enough to produce pH 6.5 |

This composition comes in the form of a serum with a pH of 6.2.

It is obtained by implementing the following stages:
Mixing A, dispersing the gel thoroughly while being stirred mechanically,
Mixing B. Heating A and B to 80° C.,
Emulsifying B in A under an emulsifying agent,
At 40° C., adding C in order, homogenizing,
Adjusting the pH with D, while being stirred mechanically,
Continuing homogenization until the serum is uniform,
Allowing stirring to continue until cooling, 30° C.

### IV. Evaluation of the Effect of Activators of Autophagy on Cellular and Tissue Longevity

### IV. 1 In-Vitro Studies on Molecular Markers of Cellular and Tissue Longevity of the Skin

1. 1. Study of the Accumulation of Lipofuscin Aggregates

The objective of this study is to evaluate the capacity of activators of autophagy to limit the formation of lipofuscin aggregates that alter cellular function and that are correlated with the reduction of cellular longevity.

This study was carried out by confocal microscopy on normal human fibroblasts subjected to moderate and repeated treatments with hydrogen peroxide (H₂O₂), making it possible to induce cellular senescence.

The operating procedure is described below.

On D0, the normal human fibroblasts are inoculated in a culture medium. The cells are then incubated at 37° C.

For several days, the fibroblasts are treated and stressed:
Controls: the human fibroblasts are inoculated in a culture medium and then treated with an H₂O₂ solution; this repeated treatment makes it possible to induce the state of cellular senescence characterized by a growth ratio and a β-galactosidase activity,
Treated: the human fibroblasts are inoculated in a culture medium in the presence of activators of skin-cell autophagy of Examples I. 1 and I. 2 at 1% (V/V) and then treated with an H₂O₂ solution.

The visualization of the lipofuscin is carried out using a microscope coupled to image analysis software. Lipofuscin manifests itself through the appearance of autofluorescent grains in the cell cytoplasm. The quantity of lipofuscin is proportional to the intensity of autofluorescence of the cells.

The results that are obtained are expressed in terms of fluorescence intensity/number of cells (UA) and are presented in the table below:

| | Effectiveness/Control |
|---|---|
| | Stressed Fibroblasts (%) |
| Stressed Control | 0 |
| 1% Prunus cerasus Extract | −35% |
| 1% Metschnikowia hawaiiensis | −45% |
| Extract | |

The cytoplasmic accumulation of lipofuscin aggregates is significantly increased when the normal human fibroblasts are subjected to a moderate and repeated H₂O₂ stress. In addition, it is noted that the activators of autophagy make it possible to reduce the accumulation of lipofuscin in comparison to control stressed fibroblasts.

1. 2. Study of the Protection of the Extracellular Matrix

The objective of this study is to evaluate the effect of activators of skin-cell autophagy on the synthesis of MMP-1, of which the synthesis is increased during aging.

The study was carried out by ELISA metering on normal human fibroblasts having undergone moderate and repeated treatments with hydrogen peroxide (H₂O₂), making it possible to induce cellular senescence.

The operating procedure is described below.

On D0, the normal human fibroblasts are inoculated in a culture medium. The cells are then incubated at 37° C.

For several days, the fibroblasts are treated and stressed:
Controls: the human fibroblasts are inoculated in a culture medium and then treated with an H₂O₂ solution; this repeated treatment makes it possible to induce the cellular senescence state characterized by a growth ratio and a β-galactosidase activity,
Treated: the human fibroblasts are inoculated in a culture medium in the presence of the activators of skin-cell autophagy of Example I. 1 at 1% (V/V) and then treated with an H₂O₂ solution.

The cellular supernatants are recovered. The MMP-1 metering is carried out using an ELISA metering kit.

The results that are obtained are presented in the table below:

| | Effectiveness/Control |
|---|---|
| | Stressed Fibroblasts (%) |
| Control | 0 |
| 1% Prunus cerasus Extract | −48% |

The synthesis of MMP-1 is significantly increased when the normal human fibroblasts are subjected to a moderate and repeated H₂O₂ stress.

In addition, it is noted that the activators of autophagy make it possible to reduce by 35% the synthesis of MMP-1 in comparison to the control stressed fibroblasts. This effect is significant and dose-dependent. The integrity of the matrix environment is thus preserved.

### IV. 2 In-Vivo Studies: Evaluation of the Effect on the Signs of Aging of Photoexposed Skin

2. 1. Study of the Anti-Wrinkle Effect on Photoexposed Skin

With age, the aging of tissue is reflected in the cutaneous area by the appearance of wrinkles and age spots.

The objective of this study is to evaluate, in vivo, the anti-wrinkle effect on photo-aged skin of an activator of skin cells (Example I. 1) formulated at 3% vs. placebo in the area of the crow's-feet and by fringe spraying.

The study was conducted on 20 healthy volunteers aged between 52 and 70 years and selected as having photoexposed skin.
The acquisitions were made using a fringe projection device dedicated to the 3D measurement of cutaneous relief (Eotech, France). The 3D acquisitions were made in the area of the crow's-foot before and after 28 days of twice-daily treatment.

The most relevant parameters adopted for this study are:
3D-roughness parameters
   Sq: Root mean square of surface roughness
   Sa: Arithmetic mean of surface roughness.
Volume parameters
   Negative Volume: volume less than the surface of the skin.

A reduction of these different parameters is characteristic of an improvement of the relief of the surface being studied and a reduction of wrinkles

The results that are obtained are presented in the table below:

| | Variation/Placebo (%) |
|---|---|
| Parameter Sa | −6.1% |
| Parameter Sq | −5.9% |
| Negative Volume | −14.2% |

Under the conditions of this study, after 28 days of twice-daily applications and in comparison to the placebo, the active ingredient of Example I. 1 formulated with 3% in emulsion:
Smoothes the cutaneous relief in the area of the crow's-feet. Actually, the activator of the tested autophagy significantly decreases the 3D roughness parameters:
   the parameter Sa of 6.1% (p=0.0040),
   the parameter Sq of 5.9% (p=0.0042),
   reduces the wrinkles by making possible a significant reduction of the negative volume parameter (−14.2%, p=0.0079).

This study therefore clearly shows that an activator of autophagy makes it possible to slow down the cutaneous aging and that it therefore increases the cellular longevity of the skin.

1. A method of increasing cellular and tissue longevity of skin, comprising administering to a subject in need thereof an effective amount of an active ingredient that activates skin-cell autophagy.

2. The method according to claim 1, wherein the active ingredient acts on molecular mechanisms involved in cellular and tissue longevity of the skin.

3. The method according to claim 1, wherein the active ingredient limits accumulation of lipofuscin in the cells of the skin.

4. The method according to claim 1, wherein the active ingredient limits degradation of the cellular matrix induced by the metalloproteinases-1.

5. The method according to claim 1, wherein the active ingredient is a synthetic molecule or an active ingredient obtained from at least one plant, one alga, or one yeast.

6. The method according to claim 1, wherein the active ingredient is obtained from the fruit-bearing peduncle of Prunus cesarus.

7. The method according to claim 6, wherein the active ingredient a hydrolyzate of the fruit-bearing peduncle of Prunus cesarus.

8. The method according to claim 1, wherein the active ingredient is obtained from Metschnikowia hawaiiensis.

9. The method according to claim 8, wherein the active ingredient is a hydrolyzate of Metschnikowia hawaiiensis.

10. The method according to claim 1, wherein the active ingredient is selected by a test that is carried out on cultures of skin cells comprising the following stages:
culturing skin cells in a culture medium,
removing the culture medium and replacement by a culture medium comprising a stressing agent inducing a nutritive and/or oxidative stress,
adding the active ingredient that is to be tested in the culture medium, and
analyzing the expression of MAP-LC3, ATG5-12, phosphorylated mTOR, phosphorylated p53 protein, the expression of the protein DRAM and/or AMPK by said cells, and comparing results from said analyzing with those obtained on cultures of skin cells that are not treated with the active ingredient that is to be tested.

11. A cosmetic skin treatment process for combating cutaneous aging, comprising topically applying on the skin of a subject in need thereof an activator of skin-cell autophagy or a composition containing an activator of skin-cell autophagy.

12. A cosmetic process for treatment of photoexposed skin to delay the appearance of aging thereof, comprising topically applying on the skin of a subject in need thereof an activator of skin-cell autophagy or a composition that contains an activator of skin-cell autophagy.
